(19) United States
(12) Patent Application Publication
(10) Pub. No.: US 2010/0069748 A1
(43) Pub. Date: Mar. 18, 2010

(54) SYRINGE-LIKE HAND SWITCH

(75) Inventor: Tetsuya Yamamoto, Osaka (JP)

Correspondence Address: MCDERMOTT WILL & EMERY LLP, 600 13TH STREET, N.W., WASHINGTON, DC, 20005-3096, US

(73) Assignee: SUGAN CO., LTD., Osaka-shi, Osaka (JP)

(21) Appl. No.: 12/521,045

(22) PCT Filed: Dec. 28, 2006
(86) PCT No.: PCT/JP2006/326237

§ 371 Date: Jun. 24, 2009

(51) Int. Cl. A61B 6/00

(52) U.S. Cl. 600/432

(57) ABSTRACT

At the insertion movement of plunger member in cylinder member, a photosensor is in the position of sensing the movement of slit area. As a result, when the plunger member is pressed inward by an operator, light transmission zones and light shielding zones alternately provided in the slit area are sensed by the photosensor. Accordingly, there can be provided a syringe-like hand switch such that in the chemical introduction control of injector head for injection of chemicals, such as a contrast medium, in patients, both parameters for injection speed and injection quantity can be controlled by the senses at use of conventional syringes.

### TECHNICAL FIELD

The present invention relates to a syringe-like hand switch employed for controlling introduction of a chemical liquid into an injector head for injecting the chemical liquid into a patient.

### BACKGROUND ART

In order to cure an ischemic heart disease according to catheterization, coronary angioplasty has recently been employed. The coronary angioplasty denotes an intravascular operation of dilating an occluded or stenosed lesion in the cardiac artery (coronary artery) by catheterization, and is referred to as percutaneous transluminal angioplasty (PCTA) or percutaneous coronary intervention (PCI).

As instruments employed for this coronary angioplasty, a balloon (balloon catheter), a mesh metal (stent), a high-speed-rotating olivary drill (rotor blater), a DCA (dee cee éi) excising an arteriosclerotic tissue by pressing a cutter and the like are employed. A cure employing each of these instruments is performed under angiography with a contrast medium injector referred to as an injector head.

While it follows that a proper injection rate is appropriately selected under the judgment of an operator in injection of a contrast media into a patient, a hand switch disclosed in International Patent Laying-Open No. WO2004-086438 (Patent Document 1) can be listed as such a one that the operator controls the injection rate of the contrast medium.

In this hand switch disclosed in Patent Document 1, the injection rate of the contrast medium is controlled by controlling the quantity of light with a photosensor technique, in order to provide an explosion-proof hand switch.

In the aforementioned hand switch, however, the injection speed for the contrast medium is controlled on the basis of the quantity of light responsive to the quantity of pressing, and the injection rate cannot be simultaneously controlled by the hand switch. The contrast medium is injected from the injector head with the hand switch mainly because the injection pressure is high and a constant contrast medium must be stably injected within a constant time.

Patent Document 1: International Patent Laying-Open No. WO2004-086438

### DISCLOSURE OF THE INVENTION

### Problems to be Solved by the Invention

A problem to be solved by the present invention resides in such a point that a conventional hand switch controls only the injection speed for a chemical liquid and cannot simultaneously control the injection rate when controlling introduction of the chemical liquid into an injector head for injecting the chemical liquid such as a contrast medium into a patient. Accordingly, an object of the present invention is to provide a syringe-like hand switch enabling control of both parameters of the injection speed and the injection rate with a sense of using a conventional syringe when controlling introduction of a chemical liquid into an injector head for injecting the chemical liquid such as a contrast medium into a patient.

### Means for Solving the Problems

The syringe-like hand switch based on the present invention is a syringe-like hand switch employed for controlling introduction of a chemical liquid into an injector head for injecting the chemical liquid into a patient, and comprises a cylinder portion provided to extend in one direction, a plunger portion provided to be pressable into and extractable from the aforementioned cylinder portion, a slit region provided on the aforementioned plunger portion to move following movement of the aforementioned plunger portion, on which light transmission regions and light shielding regions are alternately arranged, and a photosensor provided on the aforementioned cylinder portion and having light transmission means for transmitting light and photoreceiving means receiving light emitted from the aforementioned light transmission means and transmitted through the said slit region, which are opposed to each other through the aforementioned slit region.

The aforementioned slit region and the aforementioned photosensor are provided with a positioning mechanism mutually positioning the aforementioned photosensor and the aforementioned slit region so that the aforementioned photosensor is located on a position for sensing movement of the aforementioned slit region when the aforementioned plunger portion is inserted and moved into the aforementioned cylinder portion and located on a position for retreating from the aforementioned slit region when the aforementioned plunger portion is extracted from the aforementioned cylinder portion.

### EFFECTS OF THE INVENTION

According to the syringe-like hand switch based on the present invention, the photosensor is located on the position for sensing movement of the slit region when the plunger portion is inserted and moved into the cylinder portion. Consequently, it follows that the light transmission regions and the light shielding regions alternately formed on the slit region are sensed by the photosensor when the plunger portion is pressed by an operator.

When a control portion of the injector head recognizes a signal passing through the light transmission regions as “1” and a signal passing through the light shielding regions as “0” in the signal from the photosensor, the “injection rate” and the “injection speed” of the chemical liquid can be decided in correspondence to the “quantity” and the “speed” of the signal “1” passing through the light transmission regions in a unit time, for example.

Consequently, the hand switch according to the present invention has the syringe-like shape comprising the cylinder portion and the plunger portion, whereby the operator is enabled to control introduction of the chemical liquid (control the “injection rate” and the “injection speed”) into the injector head for injecting the chemical liquid such as a contrast medium into a patient with a sense of pressing a syringe.

### BRIEF DESCRIPTION OF THE DRAWINGS

FIG. 1 is an overall perspective view showing the appearance structure of a syringe-like hand switch according to a first embodiment based on the present invention.

FIG. 2 is a longitudinal sectional view taken along the line II-II in FIG. 1.

FIG. 3 is a partial plan view showing only the internal structure of the syringe-like hand switch according to the first embodiment based on the present invention.

FIG. 4 is a sectional view taken along the line IV-IV in FIG. 3.

FIG. 5 is a diagram showing a state of a signal from a photosensor in an initial state of the syringe-like hand switch according to the first embodiment based on the present invention.

FIG. 6 is a longitudinal sectional view corresponding to that taken along the line II-II in FIG. 1, showing a first pressing state of the syringe-like hand switch according to the first embodiment based on the present invention.

FIG. 7 is a longitudinal sectional view corresponding to that taken along the line II-II in FIG. 1, showing a second pressing state of the syringe-like hand switch according to the first embodiment based on the present invention.

FIGS. 8(A) and (B) are partial plan views showing only the internal structure in the state of FIG. 7, (A) shows a case where a photo switch is of a transmission type, and (B) shows a case where the photo switch is of a reflection type.

FIG. 9 is a diagram showing a state of a signal from the photosensor in a sensing state of the syringe-like hand switch according to the first embodiment based on the present invention.

FIG. 10 is an overall perspective view showing the appearance structure of a syringe-like hand switch according to a second embodiment based on the present invention.

FIG. 11 is an overall perspective view showing only the internal structure of the syringe-like hand switch according to the second embodiment based on the present invention.

FIG. 12 is a partial perspective view showing the structures of components in the syringe-like hand switch according to the second embodiment based on the present invention.

FIG. 13 is a first state diagram showing an operating state of the syringe-like hand switch according to the second embodiment based on the present invention.

FIG. 14 is a sectional view taken along the line XIV-XIV in FIG. 13.

FIG. 15 is a second state diagram showing another operating state of the syringe-like hand switch according to the second embodiment based on the present invention.

FIG. 16 is a third state diagram showing still another operating state of the syringe-like hand switch according to the second embodiment based on the present invention.

FIG. 17 is a fourth state diagram showing a further operating state of the syringe-like hand switch according to the second embodiment based on the present invention.

FIG. 18 is a fifth state diagram showing a further operating state of the syringe-like hand switch according to the second embodiment based on the present invention.

FIG. 19 is a sixth state diagram showing a further operating state of the syringe-like hand switch according to the second embodiment based on the present invention.

FIG. 20 is a first diagram showing another mode of the syringe-like hand switch according to the second embodiment based on the present invention.

FIG. 21 is a second diagram showing still another mode of the syringe-like hand switch according to the second embodiment based on the present invention.

FIG. 22 is an overall perspective view showing the appearance structure of a syringe-like hand switch according to a third embodiment based on the present invention.

FIG. 23 is a partial plan view showing only the internal structure of the syringe-like hand switch according to the third embodiment based on the present invention.

FIG. 24 is an overall exploded perspective view showing the structures of components of the syringe-like hand switch according to the third embodiment based on the present invention.

FIG. 25 is a partial exploded perspective view showing the components constituting the syringe-like hand switch according to the third embodiment based on the present invention.

FIG. 26 is a sectional view taken along the line XXVI in FIG. 23.

FIG. 27 is a sectional view taken along the line XXVII in FIG. 23.

FIG. 28 is an exploded perspective view showing the structure of a rotary block employed for the syringe-like hand switch according to the third embodiment based on the present invention.

FIG. 29 is a first state diagram showing an operating state (pressing operation) of the syringe-like hand switch according to the third embodiment based on the present invention.

FIG. 30 is a sectional view taken along the line XXX in FIG. 29.

FIG. 31 is a second state diagram showing another operating state (extracting operation) of the syringe-like hand switch according to the third embodiment based on the present invention.

FIG. 32 is a sectional view taken along the line XXXII in FIG. 31.

### DESCRIPTION OF THE REFERENCE SIGNS

1,2,3 syringe-like hand switch, 100,300,400 cylinder portion, 110 finger hook portion, 150 guide hole, 151 guide member, 152 rotating shaft, 153 pinion gear, 156 rear stopper, 157 front stopper, 180,380,480 plunger portion, 181 bar portion, 182 finger pad portion, 183 rack, 187 rotating disc, 187a retreat region, 187b slit region, 190 coil spring, 200 optical fiber cable, 201 photosensor, 201a light transmission means, 201b photoreceiving means, 301,401 cover, 350,450 base plate, 350a,450a finger hook hole, 351,351 outer sidewall, outer guide wall, 351g,351g guide protrusion, 381,481 bar portion, 381a,482 finger receiving portion, 382 guide shaft portion, 383,383 guide groove, 384,384 inner guide wall, 384a notched region, 384s guide slit, 384z support portion, 385 block, 385a through-hole, 385g guide protrusion, 386 O-ring, 387 plate, 387a light shielding region, 387b light transmission region, 451,451 outer sidewall, 451a,451a guide rail, 451b recessed groove, 451c protrusion, 451d elastic section, 452 first recessed groove, 453 second recessed groove, 454 third recessed groove, 455 fourth recessed groove, 456,457 photosensor support post, 481 thread groove, 481b,481b guide groove, 490 rotary block, 491 rotating nut, 491a first outer surface region, 491b second outer surface region, 491c third outer surface region, 491d fourth outer surface region, 491e circumferential groove, 491f positioning protrusion, 492 cylindrical guide portion, 492a thread, 493 rotating disc, 493a ring plate, 493b light shielding region, 493c light transmission region, 493d recessed notch, 493s slit region, 494 control disc, 494a shutter plate, 494b light shielding film, 494c,494c control protrusion, 494d elastic section, 501 pivoting member, 502 rotating shaft, S1,S2 space.

### BEST MODES FOR CARRYING OUT THE INVENTION

Syringe-like hand switches according to respective embodiments based on the present invention are now described with reference to the drawings. Portions identical or corresponding to each other in the respective embodiments are denoted by the same reference signs, and redundant description may not be repeated.

### First Embodiment

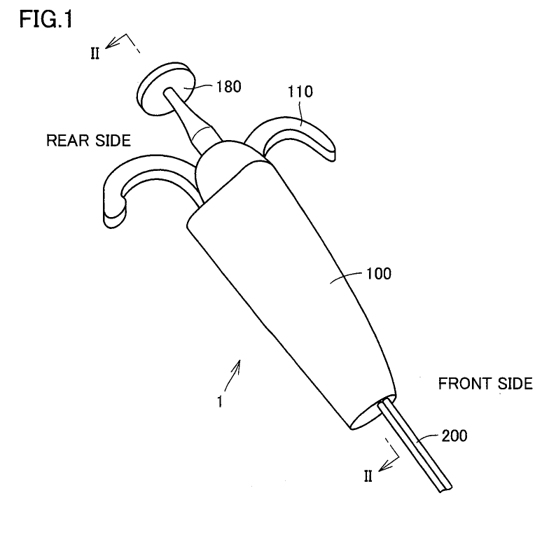

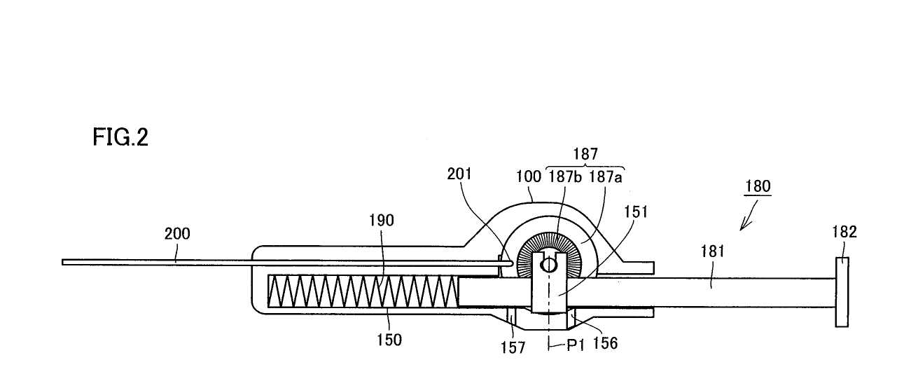

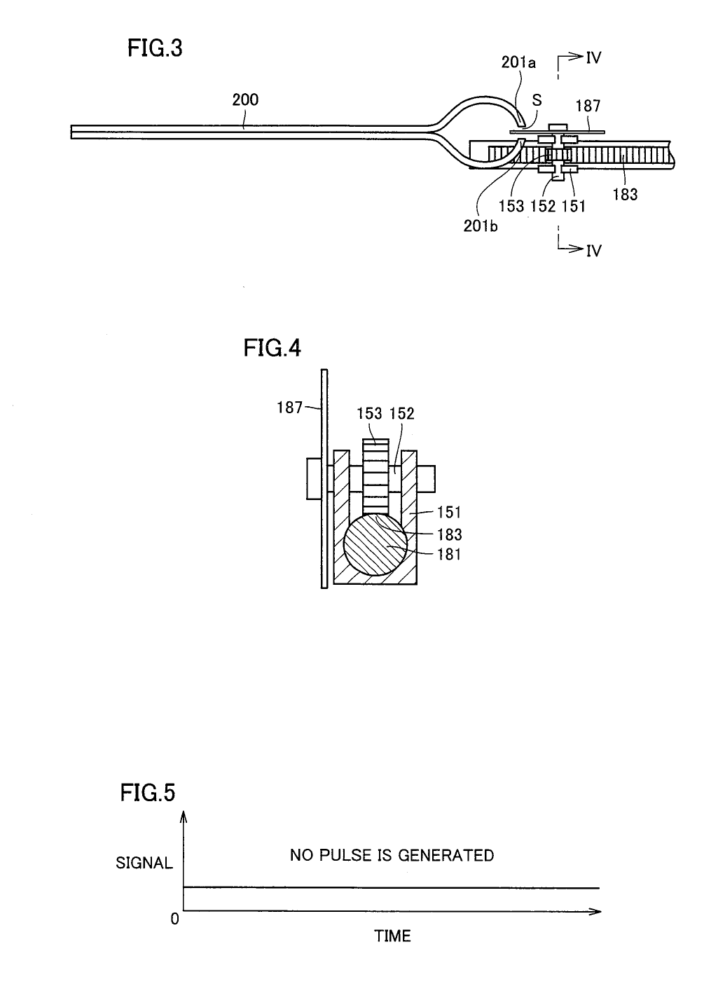

The structure of a syringe-like hand switch 1 according to this embodiment is now described with reference to FIGS. 1 to 4. FIG. 1 is an overall perspective view showing the appearance structure of syringe-like hand switch 1 according to this embodiment, and FIG. 2 is a longitudinal sectional view taken along the line II-II in FIG. 1. FIG. 3 is a partial plan view showing only the internal structure, and FIG. 4 is a sectional view taken along the line IV-IV in FIG. 3.

First, referring to FIG. 1, syringe-like hand switch 1 according to this embodiment has a syringe-like appearance shape as a whole, and includes a long and narrow cylinder portion 100 provided to extend in one direction and a plunger portion 180 provided to be pressable into and extractable from cylinder portion 100. Bent finger hook portions 110 for engaging with fingers of an operator are formed on the side of cylinder portion 100 provided with plunger portion 180. An optical fiber cable 200 for a photosensor 201 described later is extracted from the forward end side of cylinder portion 100.

The internal structure of syringe-like hand switch 1 is now described with reference to FIGS. 2 to 4. Plunger portion 180 includes a bar portion 181 and a substantially circular finger pad portion 182 provided on the rear end side of bar portion 181. Plunger portion 180 is arranged to be pressable and extractable along a guide hole 150 provided in cylinder portion 100. A coil spring 190 is internally provided on the forward end side in guide hole 150, and generates urging force in a direction for extracting plunger portion 180 from guide hole 150.

A guide member 151 is engaged with the outer peripheral surface of bar portion 181 of plunger portion 180. Guide member 151 has a gatelike longitudinal sectional shape as shown in FIG. 4, and is slidably engaged to cause constant frictional force between the same and the surface of bar portion 181. A rear stopper 156 and a front stopper 157 are arranged in syringe 100 at a prescribed interval, in order to limit the range of movement of guide member 151.

Guide member 151 is provided with a rotating shaft 152 extending in a direction orthogonal to the axial direction of bar portion 181, and a pinion gear 153 is coupled to rotating shaft 152. As shown in FIG. 3, a rack 183 meshing with pinion gear 153 is formed on the upper surface side of bar portion 181. A rotating disc 187 coinciding with the rotation center of rotating shaft 152 and performing rotational movement around this rotation center is mounted on one end side of rotating shaft 152.

As shown in FIG. 2, rotating disc 187 is provided with a slit region 187b annularly arranged around the rotation center on the rotating disc 187, and a retreat region 187a consisting of a light shielding region centering on the rotation center is annularly provided on the outer side of slit region 187b. Light transmission regions and light shielding regions are alternately arranged on slit region 187b.

As shown in FIGS. 2 and 3, cylinder portion 100 is provided with a photosensor 201 having light transmission means 201 for transmitting light and photoreceiving means 201b receiving light emitted from light transmission means 201a and transmitted through slit region 187b, which are opposed to each other through retreat region 187a and slit region 187b of rotating disc 187. A photodetection element of the photosensor itself is provided on the side of an injector head apparatus, and photosensor 201 performs only light emission and photoreception. Employment of a structure arranging the element itself is also possible as light transmission means 201a and photoreceiving means 201b. This also applies to the respective embodiments described later.

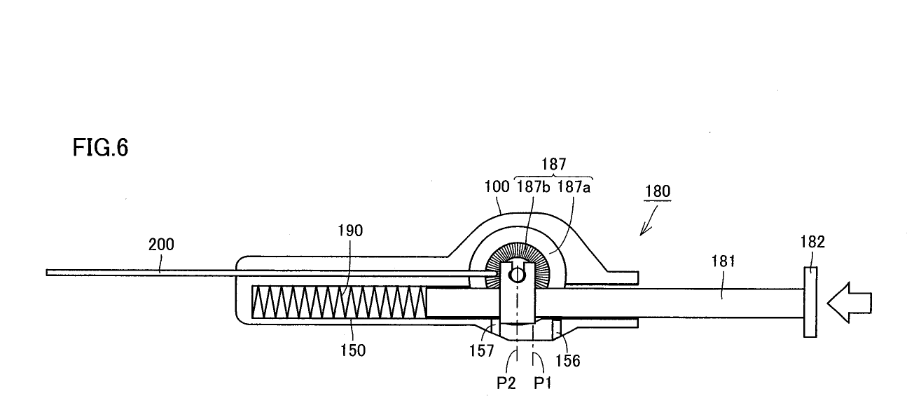

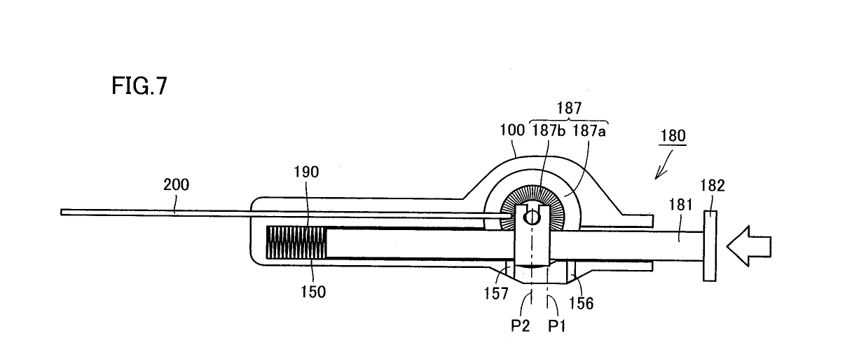

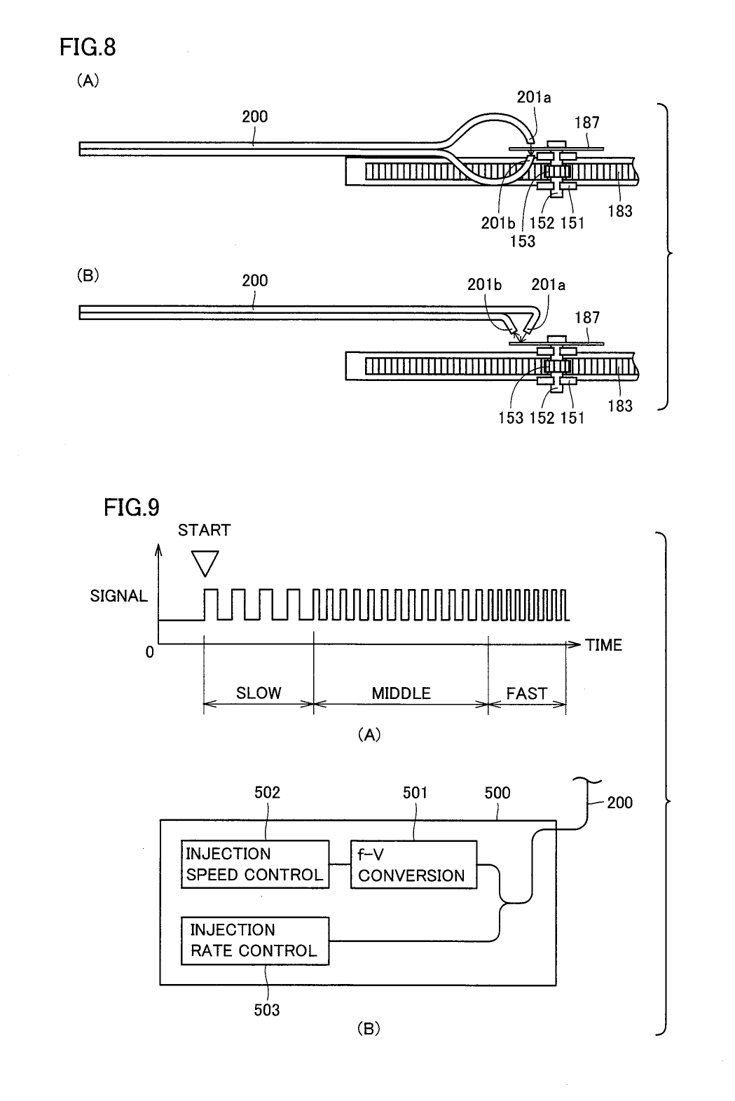

An operating mechanism of syringe-like hand switch 1 having the aforementioned structure and a positioning mechanism for mutually positioning photosensor 201 and slit region 187b are now described with reference to FIGS. 2 and 5 to 9. FIG. 5 is a diagram showing a state of a signal from photosensor 201 in an initial state, FIG. 6 is a longitudinal sectional view corresponding to that taken along the line II-II in FIG. 1, showing a first pressing state, FIG. 7 is a longitudinal sectional view corresponding to that taken along the line II-II in FIG. 1, showing a second pressing state, and FIGS. 8(A) and (B) are partial plan views showing only the internal structure in the state of FIG. 7, while (A) shows a case where photosensor 201 is of a transmission type and (B) shows a case where photosensor 201 is of a reflection type. FIG. 9 is a diagram showing an exemplary state of the signal from photosensor 201 in pressing of plunger portion 180.

First, the state shown in FIG. 2 shows a state where plunger portion 180 is most extracted. In this state, guide member 151 comes into contact with rear stopper 156, and photosensor 201 is located on a position for sensing retreat region 187a corresponding to a position for retreating from slit region 187b.

Therefore, when a control portion of an injector head recognizes a signal passing through the light transmission regions as “1” and a signal passing through the light shielding regions as “0” in the signal from photosensor 201, therefore, the signal recognized in the control portion of the injector head enters a state generating no pulse signal at all as shown in FIG. 5, and hence no chemical liquid is introduced into the injector head.

Then, when the operator presses plunger portion 180, guide member 151 advances until the same comes into contact with front stopper 157 on the basis of frictional force between the same and plunger portion 180, as shown in FIG. 6. Thus, photosensor 201 is located on a position for sensing movement of slit region 187b when plunger portion 180 is pressed and moved into cylinder portion 100. Further, it follows that rotating disc 187 rotates at a rotational frequency responsive to the pressing speed of plunger portion 180 due to the mesh between pinion gear 153 and rack 183. FIGS. 7 and 8 are diagrams showing a state where the operator most presses plunger portion 180. Coil spring 190 enters a most contracted state.

Thus, photosensor 201 is located on the position for sensing movement of slit region 187b when plunger portion 180 is pressed and moved into cylinder portion 100. Consequently, when the control portion of the injector head recognizes the signal passing through the light transmission regions as “1” and the signal passing through the light shielding regions as “0” in the signal from photosensor 201, a pulse signal shown in FIG. 9 can be recognized, for example.

Consequently, the “injection rate” and the “injection speed” of the chemical liquid can be decided correspondingly to the “quantity” and the “speed” of the signal “1” passing through the light transmission regions in a unit time. More specifically, the pulse signal shown in FIG. 9 is f-V converted, and it follows that introduction of the chemical liquid into the injector head is controlled.

When the operator terminates the introduction of the chemical liquid and separates his/her hand from plunger portion 180, plunger portion 180 is pushed back in a direction for extracting the same from cylinder portion 100 in response to the urging force of coil spring 190. At this time, guide member 151 retreats along with plunger portion 180 until the same comes into contact with rear stopper 156, on the basis of the frictional force between the same and plunger portion 180. Thus, when plunger portion 180 is extracted from cylinder portion 100, photosensor 201 is displaced from slit region 181b and located on a position for retreating to retreat region 187b. Consequently, the signal enters a state similar to the case shown in FIG. 5 and no pulse signal is generated at all when plunger portion 180 is extracted from cylinder portion 100, and hence no chemical liquid is introduced into the injector head.

Also when the movement of plunger portion 180 is stopped on an intermediate position, an operating mechanism and a positioning mechanism similar to the above function.

According to syringe-like hand switch 1 in this embodiment, photosensor 201 is located on the position for sensing movement of slit region 187b when plunger portion 180 is inserted and moved into cylinder portion 100. Consequently, it follows that photosensor 201 senses the light transmission regions and the light shielding regions alternately formed on slit region 187b when the operator presses plunger portion 180.

Syringe-like hand switch 1 has the syringe-like shape including cylinder portion 100 and plunger portion 180, whereby the operator can control introduction of the chemical liquid into the injector head (control the “injection rate” and the “injection speed”) for injecting the chemical liquid such as a contrast medium into a patient with a sense of pressing a syringe.

### Second Embodiment

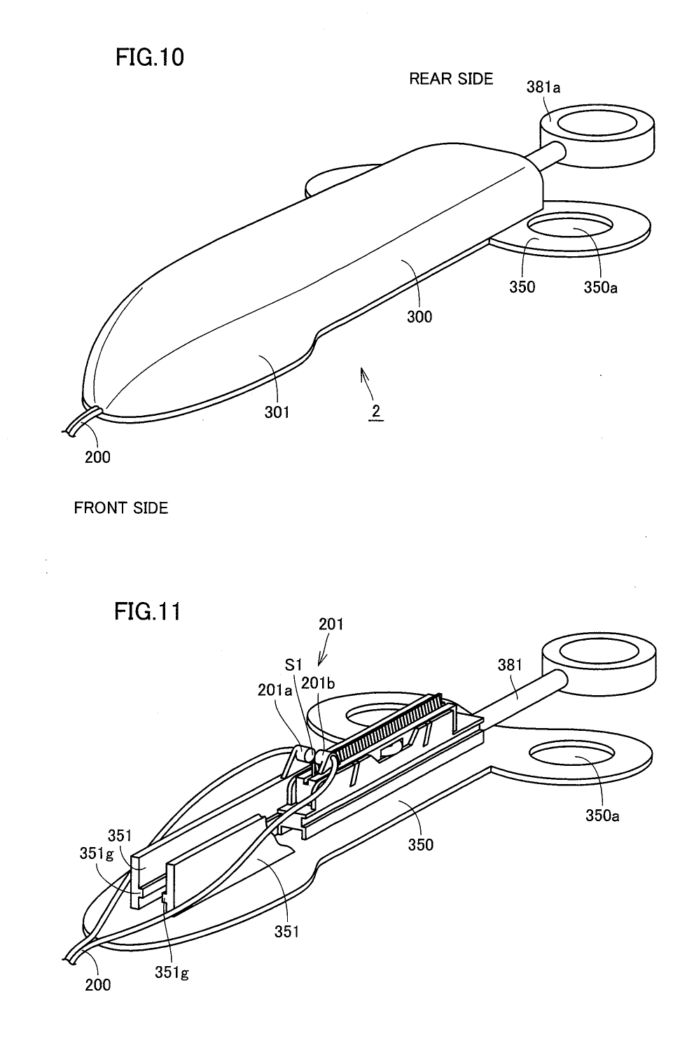

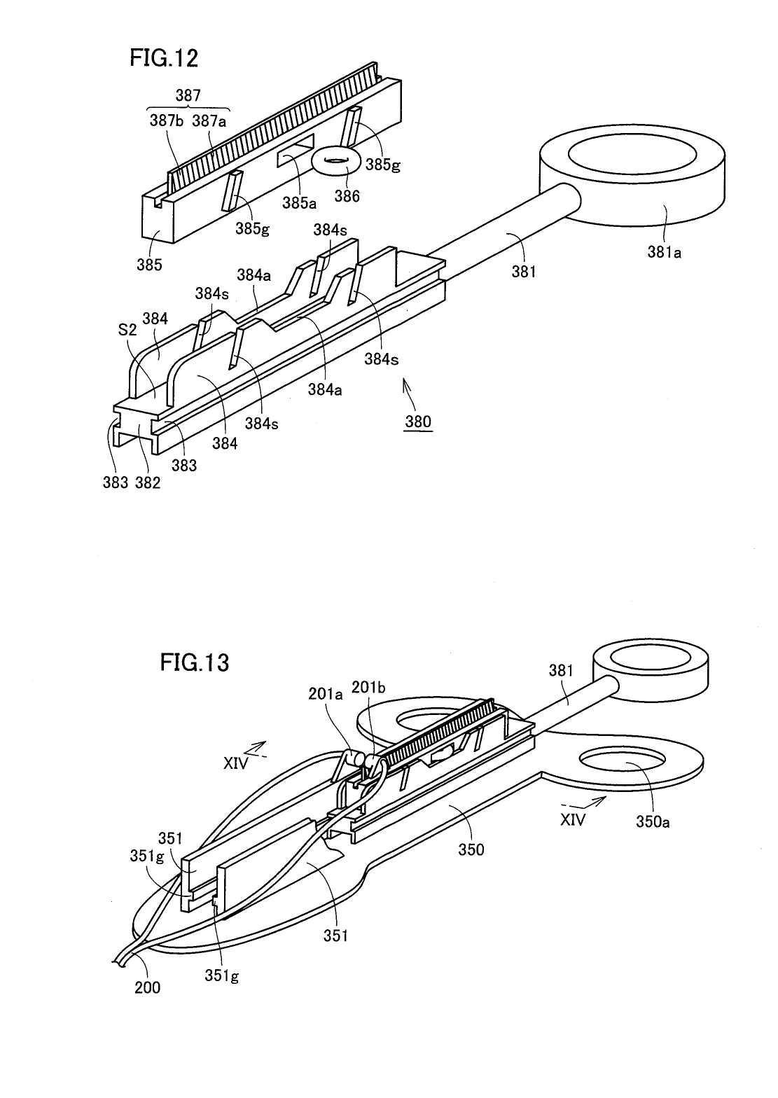

The structure of a syringe-like hand switch 2 in this embodiment is now described with reference to FIGS. 10 to 12. FIG. 10 is an overall perspective view showing the appearance structure of syringe-like hand switch 2 according to this embodiment, and FIG. 11 is an overall perspective view showing only the internal structure. FIG. 12 is a partial perspective view showing the structures of internal components.

First, referring to FIGS. 10 and 11, syringe-like hand switch 2 according to this embodiment has a syringe-like appearance shape as a whole, and includes a long and narrow cylinder portion 300 provided to extend in one direction and a plunger portion 380 provided to be pressable into and extractable from cylinder portion 300. Cylinder portion 300 includes a cover 301 and a base plate 350. Substantially circular finger hook holes 350a for engaging with fingers of an operator are provided on two portions on the side of base plate 350 provided with plunger portion 380. An optical fiber cable 200 for a photosensor 201 described later is extracted from the forward end side of cylinder portion 300.

The internal structure of syringe-like hand switch 2 is now described with reference to FIGS. 11 and 12. Outer sidewalls 351 and 351 extending in the anteroposterior direction and arranged at a prescribed interval are formed on base plate 350 of cylinder portion 300. Guide protrusions 351g and 351g extending in the anteroposterior direction are formed on the inner surfaces of the outer sidewalls on positions of a prescribed height from the lower ends.

Plunger portion 380 includes a bar portion 381 and a ring-shaped finger receiving portion 381a provided on the rear end side of bar portion 381. A guide shaft portion 382 having a gatelike cross section is provided on the forward end side of bar portion 381. Guide grooves 383 and 383 extending in the axial direction are formed on both side surfaces of guide shaft portion 382. Guide protrusions 351g provided on outer sidewalls 351 engage with guide grooves 383, thereby guiding and sliding plunger portion 380 in the anteroposterior direction.

Inner guide walls 384 and 384 extending in the axial direction are formed on the upper surface side of guide shaft portion 382 to define a guide space S2 at a constant interval. Guide slits 384s are anteroposteriorly provided on two portions of each inner guide wall 384. Guide slits 384s are provided to be inclined toward the rear end sides. A notched region 384a is provided between front and rear guide slits 384s.

A bar block 385 is arranged in guide space S2 defined by opposed inner guide walls 384 and 384. Block 385 has a substantially rectangular cross section. A plate 387 extending in the anteroposterior direction is fixed to the upper surface of block 385 in an upright state, to be along the longitudinal direction of plunger portion 380. Plate 387 constitutes a slit region, and light shielding regions 387a blocking light and light transmission regions 387b transmitting light are alternately arranged thereon. Light transmission regions 387b are provided to be inclined toward the front end sides.

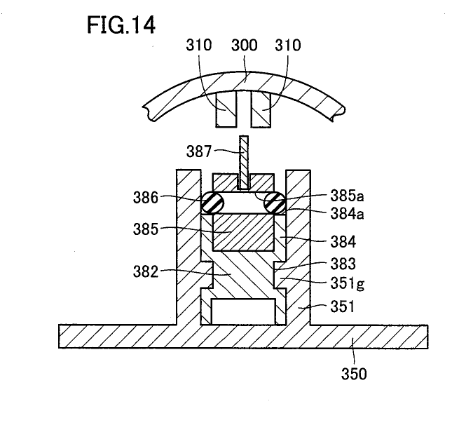

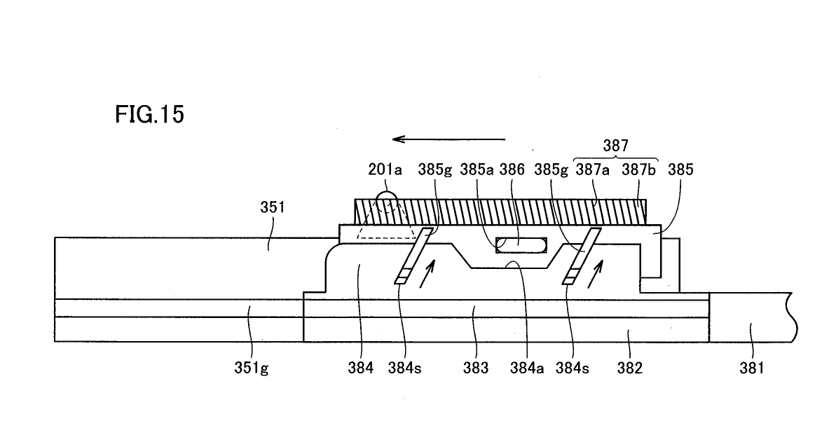

On the side surfaces of block 385, guide protrusions 385g engaging with guide slits 384s are formed on four portions in total. Transversely passing through-holes 385a are formed between front and rear guide protrusions 385g. O-rings 386 made of elastic members of rubber or a resin material engage with through-holes 385a. When O-rings 386 are inserted into through-holes 385a, O-rings 386 come into contact with the inner surfaces of outer guide walls 351 (described later) provided on base plate 350 to generate constant frictional force, as shown in FIG. 14.

As shown in FIG. 11, photosensor 201 having light transmission means 201a for transmitting light and photoreceiving means 201b receiving light emitted from light transmission means 201a and transmitted through plate 387, which are opposed to each other to hold plate 387 therebetween, is provided on an intermediate region between outer guide walls 351 of cylinder portion 300.

An operating mechanism of syringe-like hand switch 2 having the aforementioned structure and a positioning mechanism for mutually positioning photosensor 201 and plate 387 are now described with reference to FIGS. 13 to 19. FIG. 13 is a first state diagram showing an operating state of syringe-like hand switch 2, and FIG. 14 is a sectional view taken along the line XIV-XIV in FIG. 13. FIGS. 15 to 19 are diagrams showing second to sixth states showing operating states of syringe-like hand switch 2.

First, the state shown in FIG. 13 shows a state where plunger portion 380 is most extracted. In this state, photosensor 201 is positioned above plate 387, and located on a position for sensing a retreat region corresponding to a position for retreating from plate 387.

Therefore, a control portion of an injector head enters a state of regularly receiving a photoreceiving signal in relation to a signal from photosensor 201 dissimilarly to the signal waveform shown in FIG. 5, and hence no chemical liquid is introduced into the injector head.

Then, when an operator presses plunger portion 380, block 385 is inhibited from advancing on the basis of the frictional force between O-rings 386 and outer guide walls 351, as shown in FIGS. 14 and 15. However, inner guide walls 384 and 384 advance due to the pressing of plunger portion 380 by the operator. Consequently, it follows that block 385 moves up on the basis of mesh relation between guide slits 384s and guide protrusions 385g.

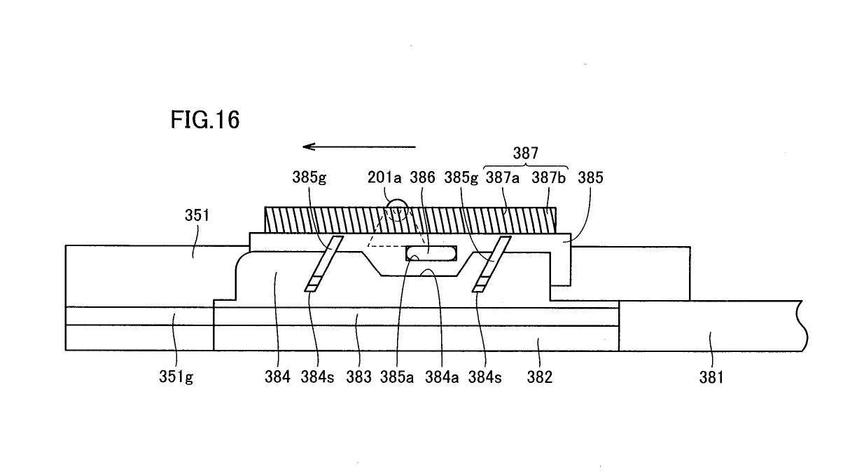

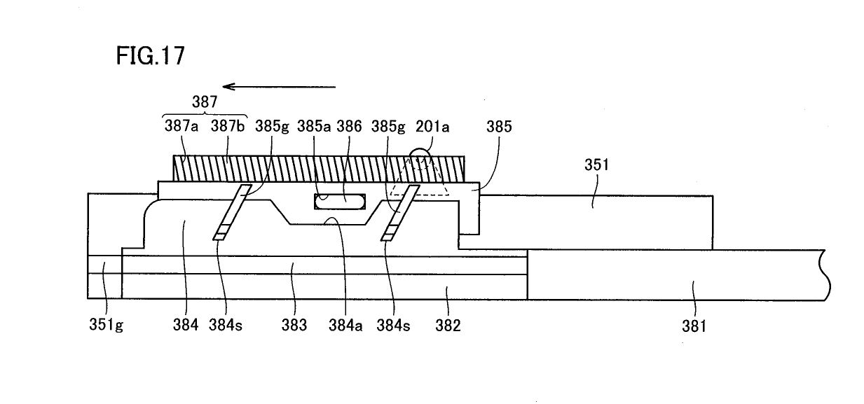

Thus, when plunger portion 380 is pressed and moved into cylinder portion 100, photosensor 201 is located on a position for sensing movement of plate 387. Further, it follows that plunger portion 380 moves at a speed responsive to the pressing speed of the operator. FIGS. 16 and 17 are diagrams showing a state where the operator presses plunger portion 380, and FIG. 17 shows a state where the operator most presses plunger portion 380.

Guide slits 384s and guide protrusions 385g, provided to be inclined toward the rear end sides respectively, do not uprightly move up but move up toward the rear end sides also when block 385 moves up. If light transmission regions 387b provided on plate 387 are uprightly provided in the vertical direction, it follows that photosensor 201 senses plurality of light transmission regions 387b when plate 387 moves up, and the signal may be disturbed. Therefore, light transmission regions 387b are provided to be inclined toward the front end sides, in order to prevent disturbance of the signal resulting from upward movement of plate 387.

The control portion of the injector head recognizes the pulse signal shown in FIG. 9 described in the aforementioned first embodiment in relation to signal input from photosensor 201, and it follows that introduction of a chemical liquid into the injector head is controlled.

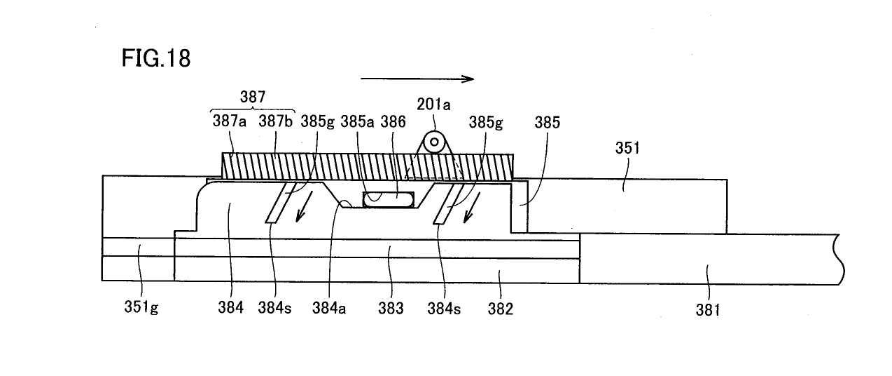

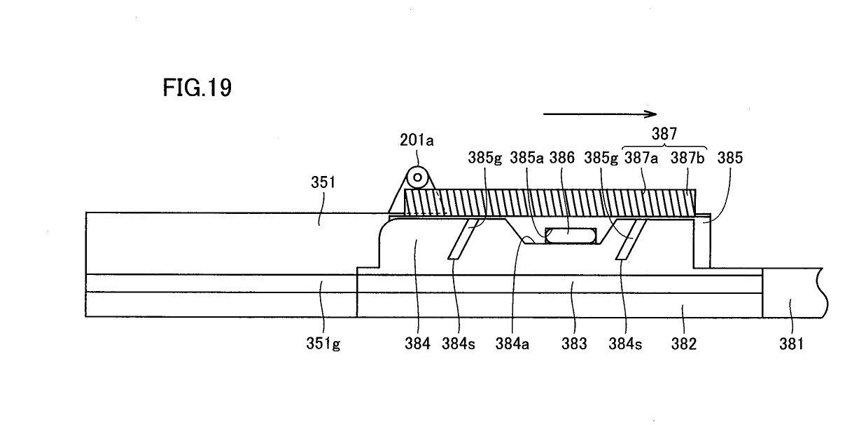

A case where the operator terminates the introduction of the chemical liquid and extracts plunger portion 380 is now described with reference to FIGS. 18 and 19. When the operator extracts plunger portion 380, block 385 is inhibited from retreating on the basis of the frictional force between O-rings 386 and outer guide walls 351. However, inner guide walls 384 and 384 retreat due to the extraction of plunger 380 by the operator. Consequently, it follows that block 385 moves down on the basis of the mesh relation between guide slits 384s and guide protrusions 385g.

Thus, photosensor 201 is displaced from plate 387 and located on a position for retreating to the retreat region. Consequently, the photoreceiving signal is regularly received when plunger portion 380 is extracted from cylinder portion 100, and hence no chemical liquid is introduced into the injector head.

Also when the movement of plunger 380 is stopped on an intermediate position, an operating mechanism and a positioning mechanism similar to the above function.

According to syringe-like hand switch 2 in this embodiment, photosensor 201 is located on the position for sensing movement of plate 387 when plunger portion 380 is inserted and moved into cylinder portion 300. Consequently, it follows that photosensor 201 senses light shielding regions 387a and light transmission regions 387b alternately formed on plate 387 when the operator presses plunger portion 380.

Further, syringe-like hand switch 2 has the syringe-like shape including cylinder portion 300 and plunger portion 380, whereby the operator can control introduction of the chemical liquid into the injector head (control the “injection rate” and the “injection speed”) for injecting the chemical liquid such as a contrast medium into a patient with a sense of pressing a syringe.

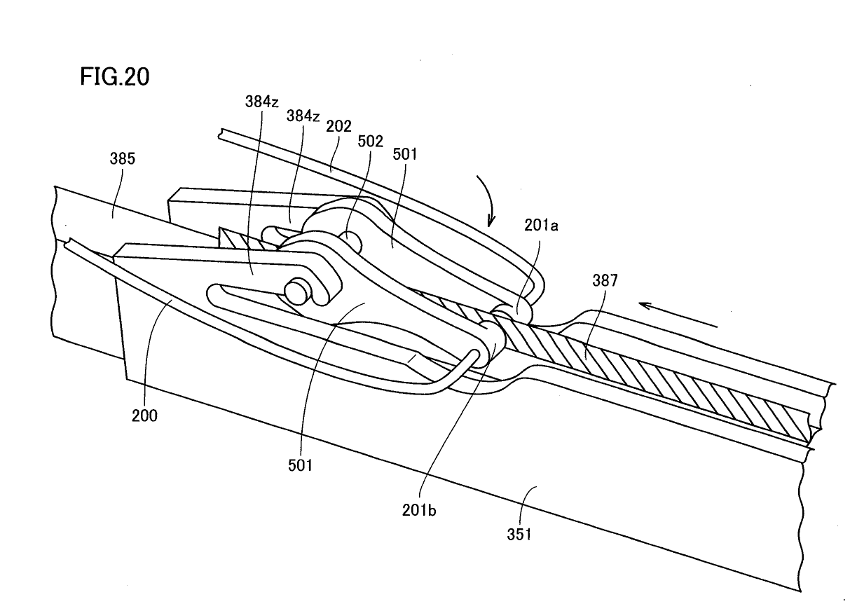

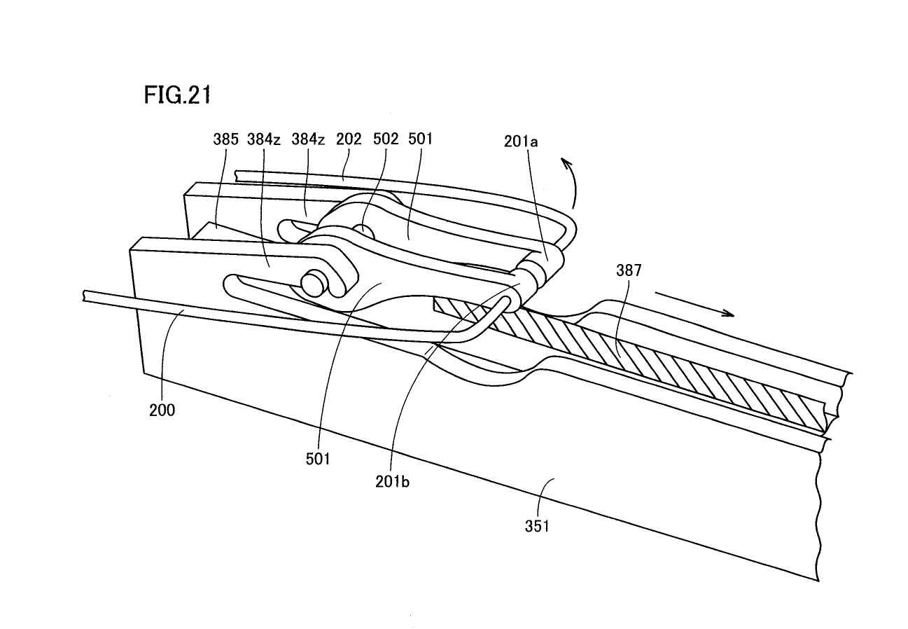

While photosensor 201 works the mechanism of moving block 385 up or down on the basis of the mesh relation between guide slits 384s and guide protrusions 385g as the mechanism for sensing movement of plate 387 when plunger portion 380 is inserted and moved into cylinder portion 300 in the aforementioned embodiment, employment of the mechanism of moving block 385 up or down can be rendered unnecessary by employing a mechanism shown in FIGS. 20 and 21 in place of this mechanism.

First, plate 387 is arranged to be uprighted on the upper surface of block 385 integrally formed on bar portion 381, as shown in FIG. 20. Support portions 384z are provided on the forward end sides of outer guide walls 351 respectively, and a rotating shaft 502 arranged in a direction orthogonal to plate 387 are rotatably pivotally supported by support portions 384z. A pivoting member 501 extending toward the rear end side, holding photosensor 201 on the forward end side thereof and provided to come into contact with the surface of block 385 around the axis of rotating shaft 502 is coupled to rotating shaft 502.

As shown in FIG. 20, a photosensor holding portion of pivoting member 501 is pivoted and urged toward the surface of block 385 on the basis of contact between pivoting member 501 and the surface of block 385 when plunger portion 380 is inserted and moved into cylinder portion 300, whereby photosensor 201 is located on the position for sensing movement of plate 387, and it follows that introduction of the chemical liquid into the injector head is controlled.

When plunger portion 380 is extracted from cylinder portion 300, on the other hand, the photosensor holding portion of pivoting member 501 is pivoted in a direction for separating from the surface side of block 385 on the basis of the contact between pivoting member 501 and the surface of block 385 as shown in FIG. 21, whereby photosensor 201 is displaced from plate 387 and located on the position for retreating to the retreat region. Consequently, the photoreceiving signal is regularly received when plunger portion 380 is extracted from cylinder portion 100, and hence no chemical liquid is introduced into the injector head.

### Third Embodiment

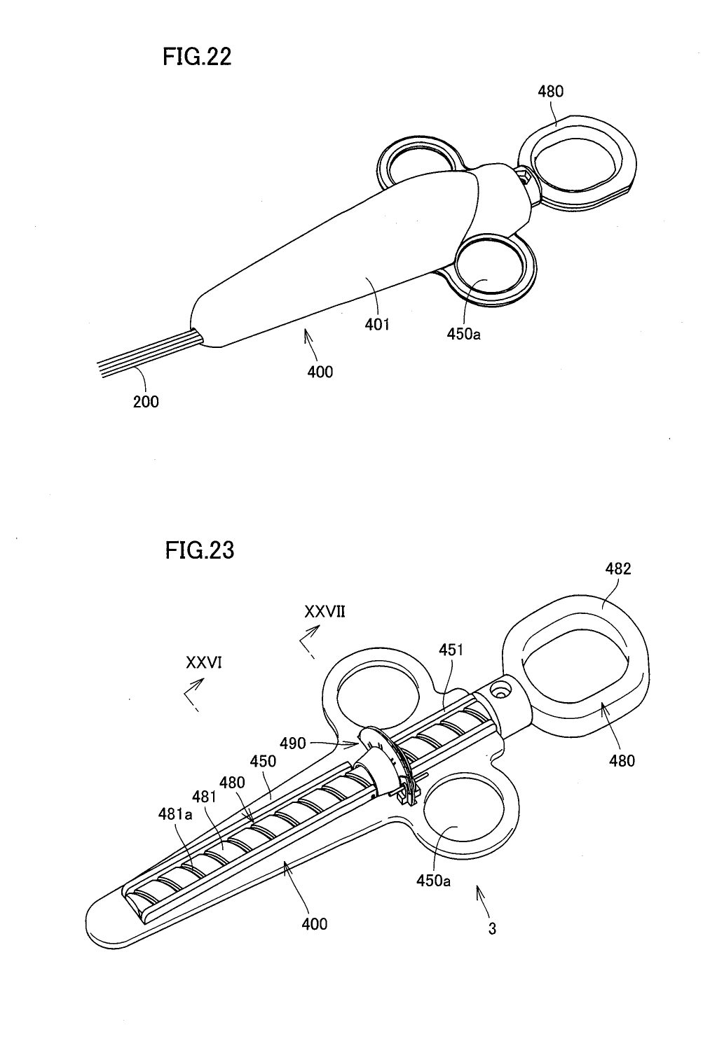

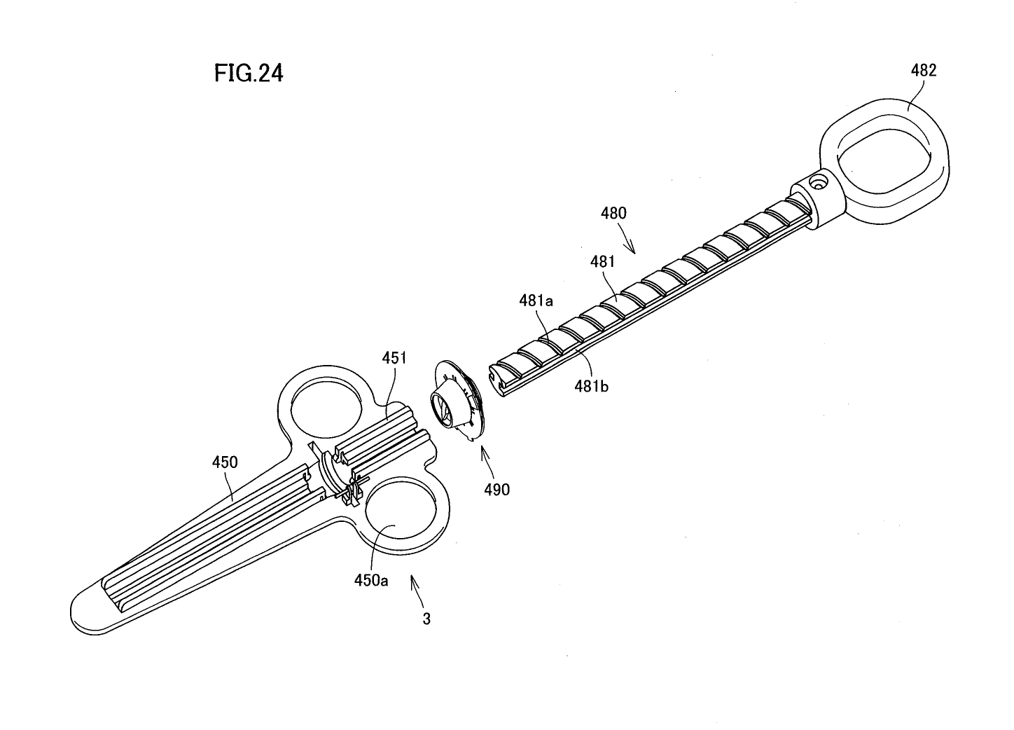

The structure of a syringe-like hand switch 3 according to this embodiment is now described with reference to FIGS. 22 to 24. FIG. 22 is an overall perspective view showing the appearance structure of syringe-like hand switch 3 according to this embodiment, FIG. 23 is a partial plan view showing only the internal structure of the syringe-like hand switch according to this embodiment, and FIG. 24 is an overall exploded perspective view showing components constituting syringe-like hand switch 3 according to this embodiment.

Syringe-like hand switch 3 according to this embodiment has a syringe-like appearance shape as a whole, and includes a long and narrow cylinder portion 400 provided to extend in one direction and a plunger portion 480 provided to be pressable into and extractable from cylinder portion 400. Cylinder portion 400 includes a cover 401 and a base plate 450. Substantially circular finger hook holes 450a for engaging with fingers of an operator are formed on two portions on the side of base plate 450 provided with plunger portion 480. An optical fiber cable 200 for a photosensor 201 described later is extracted from the forward end side of cylinder portion 400.

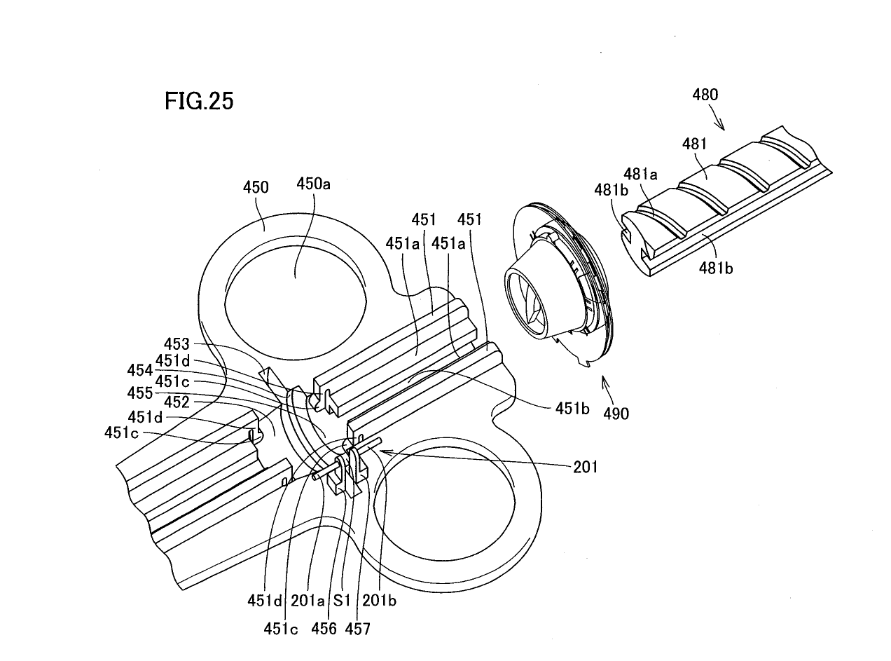

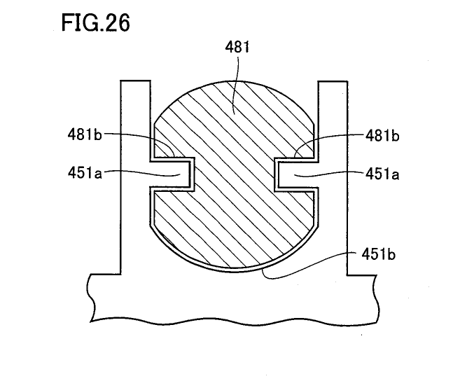

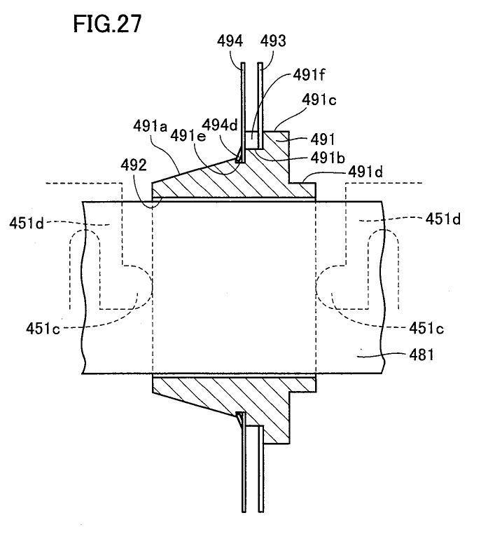

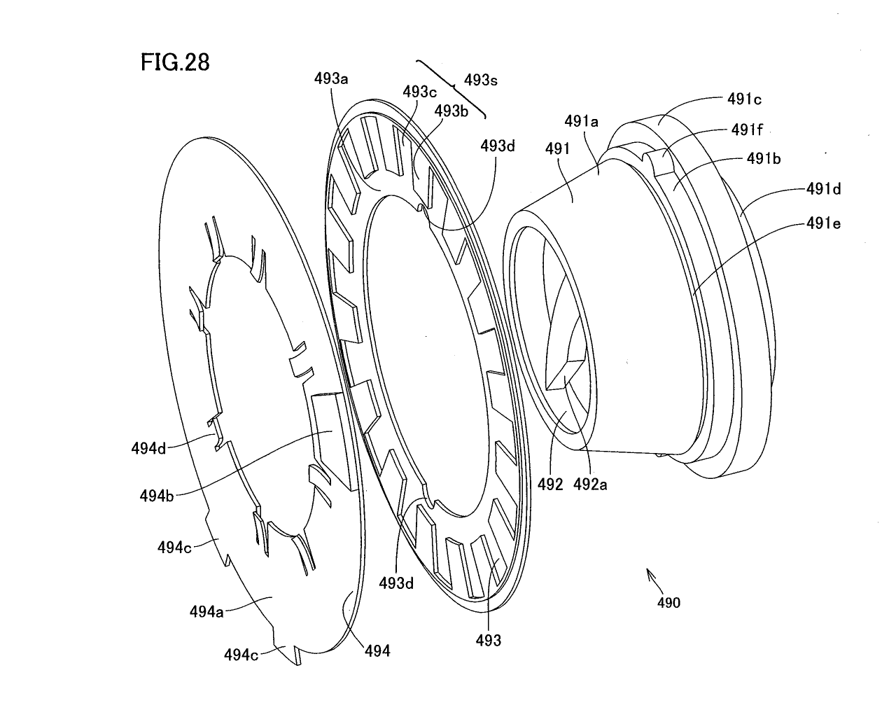

The internal structure of syringe-like hand switch 3 is now described with reference to FIGS. 24 to 28. FIG. 25 is a partial exploded perspective view showing the components constituting syringe-like hand switch 3 according to this embodiment, FIG. 26 is a sectional view taken along the line XXVI in FIG. 23, FIG. 27 is a sectional view taken along the line XXVII in FIG. 23, and FIG. 28 is an exploded perspective view showing the structure of a rotary block 490.

Plunger portion 480 includes a bar portion 481 and a ring-shaped finger receiving portion 482 provided on the rear end side of bar portion 481. Thread grooves 481 are spirally provided on the surface of bar portion 481, and guide grooves 481b and 481b extending in the axial direction are provided on both side surfaces.

Outer sidewalls 451 and 451 extending in the anteroposterior direction and arranged at a prescribed interval are formed on base plate 450 of cylinder portion 400. A recessed groove 451b along the outer surface shape of bar portion 481 of plunger portion 480 is provided on base plate 450 held between both outer sidewalls 451 and 451. Guide rails 451a and 451a extending inside are formed on intermediate portions of both outer sidewalls 451 and 451. Guide rails 451a and 451a so engage with guide grooves 481b and 481b provided on bar portion 481 of plunger portion 480 that plunger portion 480 can slide along the axial direction of bar portion 481 with respect to base plate 450.

In order to adjust insertion strength of plunger portion 480, a certain degree of coefficient of friction is preferably provided between plunger portion 480 and guide grooves 481b and 481b. A proper coefficient of friction can be generated by interposing rubber members or the like between guide grooves 481b and 481b and guide rails 451a and 451a, for example. The structure of inserting an elastic member such as the coil spring into the forward end portion of plunger portion 480 employed in the aforementioned first embodiment can also be employed. Thus, the optimum operating sense for plunger portion 480 is obtained, and fine position control of plunger portion 480 can be implemented.

In regions of outer sidewalls 451 and 451 of base plate 450 close to finger hook holes 450a, a first recessed groove 452, a second recessed groove 453, a third recessed groove 454 and a fourth recessed groove 455 are provided in a direction intersecting with the extensional direction of outer sidewalls 451 and 451, in order to allow pivoting of rotary block 490 described later. These grooves may have any shapes, unless the same exert influence on pivoting of rotary block 490.

Elastic sections 451d are provided on end surface portions of outer sidewalls 451 and 451 facing the aforementioned grooves, and protrusions 451c are provided on the forward end portions of elastic sections 451d. As shown in FIG. 27, protrusions 451c of elastic sections 451d so come into contact with both end surface portions of a rotating nut 491 described later that rotary block 490 is inhibited from movement in the anteroposterior direction and rotatably positioned.

Photosensor support posts 456 and 457 are opposed and arranged on the surface of base plate 450 to hold second recessed groove 453 therebetween, light transmission means 201a (an end surface of the optical fiber cable) of photosensor 201 is arranged on photosensor support post 456, and photoreceiving means 201b (another end surface of the optical fiber cable) is arranged on photosensor support post 457.

Rotary block 490 has a rotating nut 491, a rotating disc 493 and a control disc 494, as shown in FIG. 28. Rotating nut 491 includes a cylindrical guide portion 492 through which bar portion 481 of plunger portion 480 passes, and a thread 492a meshing with thread grooves 481 provided on bar portion 481 is spirally provided on the inner surface of cylindrical guide portion 492.

A first outer surface region 491a consisting of an inclined surface, a second outer surface region 491b consisting of a flat surface, a third outer surface region 491c consisting of a flat surface and having a larger diameter than second outer surface region 491b and a third outer surface region 491d consisting of a flat surface and having a smaller diameter than third outer surface region 491c are provided on the outer surface of rotating nut 491. A circumferential groove 491e is provided between first outer surface region 491a and second outer surface region 491b. Positioning protrusions 491f are provided on prescribed positions (two portions on positions opposed to each other by 180 degrees in this embodiment) of second outer surface region 491b.

Rotating disc 493 has an annular ring plate 493a formed by a transparent member, and an annular slit region 493s on which light transmission regions 493c and light shielding regions 493b made of the body of ring plate 493a are alternately provided is formed on ring plate 493a. Recessed notches 493d engaging with positioning protrusions 491f of second outer surface region 491b are provided on two portions of the inner diametral portion of ring plate 493a.

Control disc 494 also has an annular shutter plate 494a formed by a transparent member similarly to rotating disc 493, and a light shielding film 494b forming a non-transmission region is arranged on a prescribed position. Therefore, the remaining region other than that provided with light shielding film 494b constitutes a transmission region. Control protrusions 494c and 494c are provided on the outer peripheral surface of shutter plate 494a at a prescribed interval in the circumferential direction, while a plurality of outwardly warping elastic sections 494d are provided on the inner peripheral surface of shutter plate 494a at prescribed pitch intervals.

If control disc 494 has such a structure that only a region corresponding to light shielding film 494b forms a non-transmission region by protruding outward in the radial direction and covering slit region 493s of rotating disc 493 and the remaining region does not cover slit region 493s of rotating disc 493, shutter plate 494a may not necessarily be constituted of a transparent member.

In rotary block 490 constituted of the aforementioned structure, rotating disc 493 is arranged on second outer surface region 491b of rotating nut 491, and recessed notches 493d of ring plate 493a engage with positioning protrusions 491f, thereby positioning rotating disc 493 and fixing the position in the rotational direction, as shown in FIG. 27. Control disc 494 is arranged in circumferential groove 491e of rotating nut 491, and elastic sections 494d urge control disc 494 toward the inner wall surface of circumferential groove 491e on the basis of elastic force (frictional force) thereof.

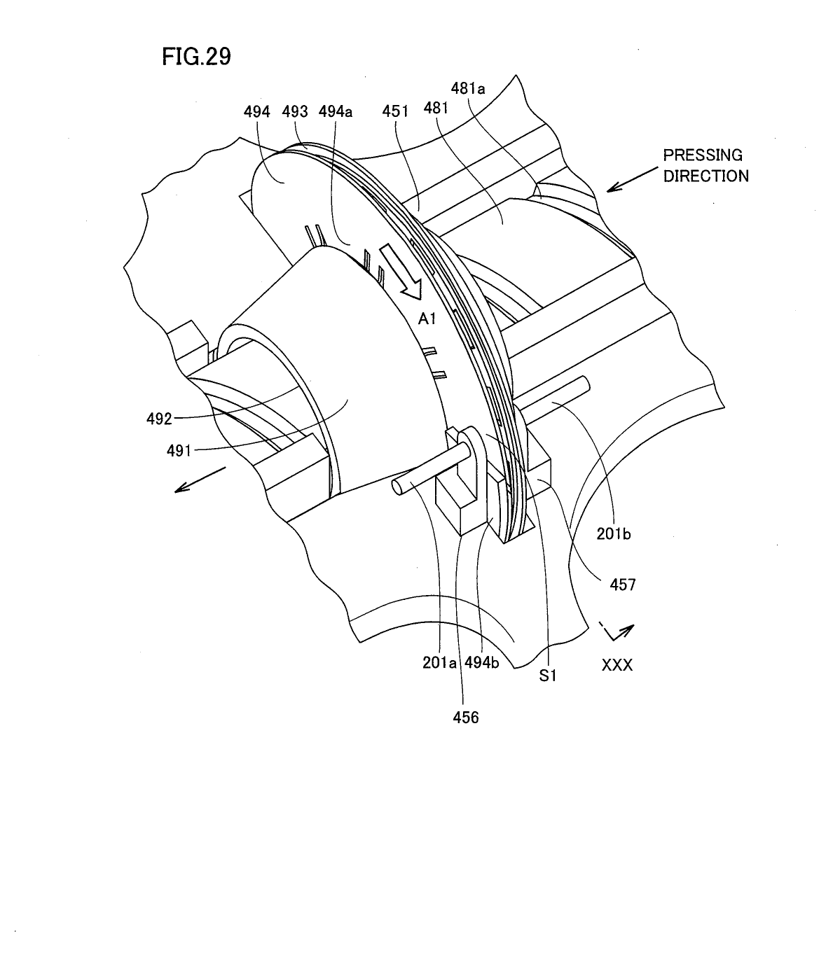

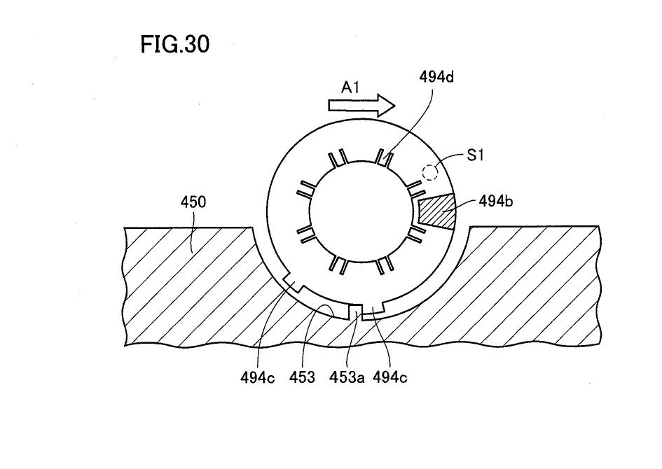

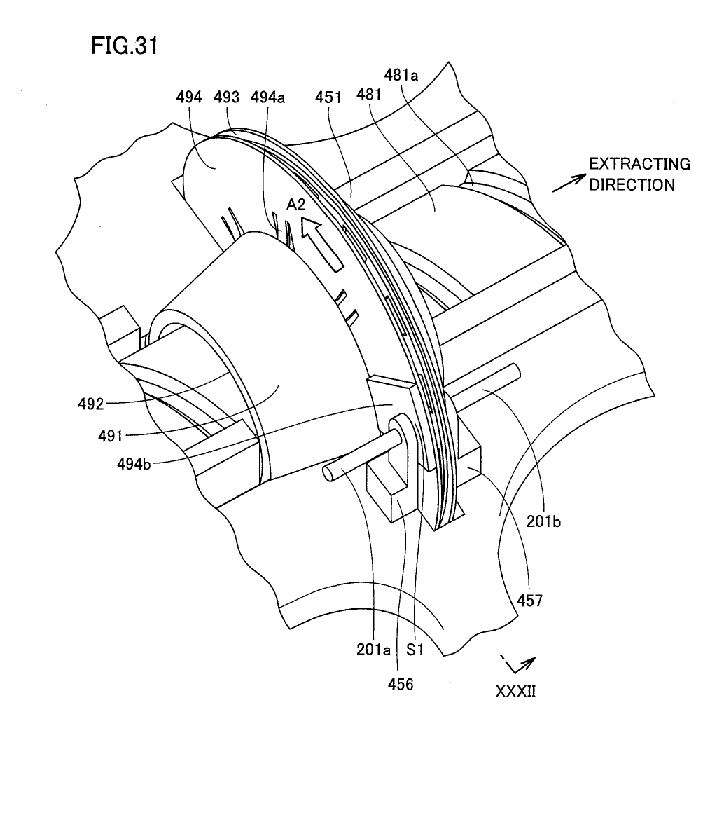

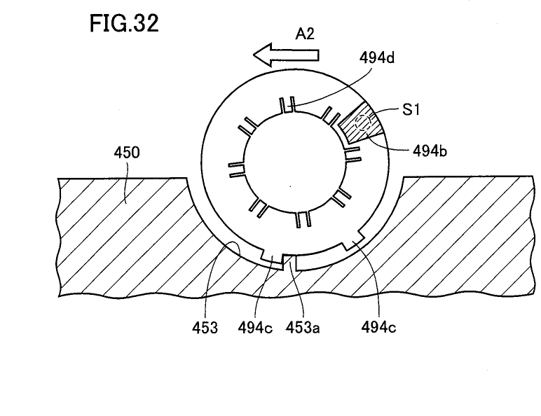

An operating mechanism of syringe-like hand switch 3 constituted of the aforementioned structure and a positioning mechanism for mutually positioning photosensor 201 and rotary block 490 are described with reference to FIGS. 29 to 32. FIG. 29 is a first state diagram showing an operating state (pressing operation) of syringe-like hand switch 3, and FIG. 30 is a sectional view taken along the line XXX in FIG. 29. FIG. 31 is a second state diagram showing another operating state (extracting operation) of syringe-like hand switch 3, and FIG. 32 is a sectional view taken along the line XXXII in FIG. 31.

First, when an operator presses plunger portion 480, rotary block 490 rotates along arrow A1 in the groove, as shown in FIGS. 29 and 30. Rotating disc 493 rotates in response to the speed of the operator pressing plunger portion 480. On the other hand, control disc 494 rotates on the basis of the elastic force (frictional force) of elastic sections 494d coming into contact with the inner wall surface of circumferential groove 491e, while one control protrusion 494c comes into contact with a stopper 453a provided on the bottom portion of second recessed groove 453, to inhibit the rotation of control disc 494. Therefore, only rotating disc 493 continues the rotation due to the pressing of plunger portion 480. At this time, light shielding film 494b of control disc 494 is located on a position for retreating from a sensor position S1 of photosensor 201, and photosensor 201 senses slit region 493s on the basis of the rotation of rotating disc 493.

When the operator extracts plunger portion 480, rotary block 490 rotates along arrow A2 in the groove, as shown in FIGS. 31 and 32. Rotating disc 493 rotates in response to the speed of the operator extracting plunger portion 480. On the other hand, control disc 494 rotates on the basis of the elastic force (frictional force) of elastic sections 494d with respect to the inner wall surface of circumferential groove 491e, while other control protrusion 494c comes into contact with stopper 453a provided on the bottom portion of second recessed groove 453 to inhibit the rotation of control disc 494. Therefore, only rotating disc 493 continues the rotation due to the extraction of plunger portion 480. At this time, light shielding film 494b of control disc 494 is located on a position shielding sensor position S1 of photosensor 201 from light, and photosensor 201 does not sense slit region 493s.

According to syringe-like hand switch 3 in this embodiment, photosensor 201 is located on the position for sensing movement of slit region 493s when plunger portion 480 is inserted and moved into cylinder portion 400. Consequently, it follows that photosensor 201 senses the light transmission regions and the light shielding regions alternately formed on slit region 493s when the operator presses plunger portion 480.

Further, syringe-like hand switch 3 has the syringe-like shape including cylinder portion 400 and plunger portion 480, whereby the operator can control introduction of a chemical liquid into an injector head (control the “injection rate” and the “injection speed”) for injecting the chemical liquid such as a contrast medium into a patient with a sense of pressing a syringe, similarly to the aforementioned first and second embodiments.

While the respective embodiments based on the present invention have been described, the embodiments disclosed this time must be considered as illustrative in all points and not restrictive. The range of the present invention is shown by the scope of claims for patent, and it is intended that all modifications within the meaning and range equivalent to the scope of claims for patent are included.

1. A syringe-like hand switch employed for controlling introduction of a chemical liquid into an injector head for injecting the chemical liquid into a patient, comprising:
a cylinder portion provided to extend in one direction;
a plunger portion provided to be pressable into and extractable from said cylinder portion;
a slit region provided on said plunger portion to move following movement of said plunger portion, on which light transmission regions and light shielding regions are alternately arranged; and
a photosensor provided on said cylinder portion and having light transmission means for transmitting light and photoreceiving means receiving light emitted from said light transmission means and transmitted through said slit region, which are opposed to each other through said slit region, wherein
said slit region and said photosensor are provided with a positioning mechanism mutually positioning said photosensor and said slit region so that said photosensor is located on a position for sensing movement of said slit region when said plunger portion is inserted and moved into said cylinder portion and located on a position for retreating from said slit region when said plunger portion is extracted from said cylinder portion.

2. The syringe-like hand switch according to claim 1, having a rotating disc rotating and moving around the rotation center of said plunger portion following movement of said plunger portion, wherein
said slit region is annularly arranged around the rotation center on said rotating disc, and
said positioning mechanism is so provided that said photosensor is located on the position for sensing movement of said slit region when said plunger portion is inserted and moved into said cylinder portion and located on the position for retreating from said slit region when said plunger portion is extracted from said cylinder portion.

3. The syringe-like hand switch according to claim 2, wherein
said slit region is annularly provided on the rotation center side on said rotating disc, and a retreat region consisting of a light shielding region centering on the rotation center is annularly provided on the outer side of said slit region, and
as to said positioning mechanism, said photosensor is located on the position for sensing movement of said slit region when said plunger portion is inserted and moved into said cylinder portion and located on a position for sensing movement of said retreat region when said plunger portion is extracted from said cylinder portion.

4. The syringe-like hand switch according to claim 2, wherein
said slit region is annularly provided on the rotation center side on said rotating disc,
said positioning mechanism has a control disc arranged concentrically with said rotating disc and having a transmission region and a non-transmission region,
the transmission region is so positioned with respect to said photosensor that said control disc is on such a position that said photosensor senses movement of said slit region when said plunger portion is inserted and moved into said cylinder portion, and
the non-transmission region is so positioned with respect to said photosensor that said control disc is on such a position that sensing of said slit region by said photosensor is inhibited when said plunger portion is extracted from said cylinder portion.

5. The syringe-like hand switch according to claim 1, wherein
said slit region is provided along the longitudinal direction of said plunger portion, and
said positioning mechanism is so provided that said photosensor is located on the position for sensing movement of said slit region when said plunger portion is inserted and moved into said cylinder portion and located on the position for retreating from said slit region when said plunger portion is extracted from said cylinder portion.

6. The syringe-like hand switch according to claim 5, wherein
said positioning mechanism is so provided that said slit region moves up to a sensing region of said photosensor following movement of said plunger portion when said plunger portion is inserted and moved into said cylinder portion and said slit region moves down to be displaced from the sensing region of said photosensor when said plunger portion is extracted from said cylinder portion.
